(19) United States
(12) Patent Application Publication
(10) Pub. No.: US 2011/0237944 A1
(43) Pub. Date: Sep. 29, 2011

(54) METHODS AND DEVICES FOR TRANSFERING CONFIGURATION SETTINGS

(75) Inventors: Guoqi SHI, Shenzhen (CN); Qiang LI, Shenzhen (CN); Yanmei GUO, Shenzhen (CN); Qinglin TAO, Shenzhen (CN); Jieying LIN, Shenzhen (CN)

(73) Assignee: SHENZHEN MINDRAY BIO-MEDICAL ELECTRONICS CO., LTD., Shenzhen (CN)

(21) Appl. No.: 13/047,588

(22) Filed: Mar. 14, 2011

(30) Foreign Application Priority Data
Mar. 26, 2010 (CN) 201010137213.0

(51) Int. Cl. A61B 8/00

(52) U.S. Cl. 600/437

(57) ABSTRACT

A method for transferring configuration settings and/or user maintenance items includes allowing a user to select a set of configuration settings and/or user maintenance items stored in a removable memory device coupled to a medical device; displaying the selected set of configuration settings and/or user maintenance items on a display device; and importing configuration settings and/or user maintenance items stored in the removable memory device to the medical device.

### CROSS-REFERENCE TO RELATED APPLICATION(S)

This application claims the benefit of Chinese Patent Application No. 201010137213.0, filed on Mar. 26, 2010, which is incorporated herein by reference.

### TECHNICAL FIELD

The present disclosure relates to medical devices, such as patient monitors and diagnostic ultrasound devices.

### BRIEF DESCRIPTION OF THE DRAWINGS

FIG. 1 is a flowchart of a process for exporting a configuration.

FIG. 2 is a flowchart of a process for importing a configuration.

FIG. 3 is a flowchart of a process for loading configuration settings.

FIG. 4 is an interface for changing configuration settings.

FIG. 5 is a block diagram of a patient monitor.

### DETAILED DESCRIPTION

When a patient monitor is used, it must be configured for specific situations. In different departments, e.g., Operating Room (OR) or Intensive Care Unit (ICU), the configuration settings of a patient monitor must be different. Even in the same department, the configuration settings of the patient monitor must be different according to different users. In order to meet the requirements of different situations and users, the patient monitor may provide the function of saving a plurality of sets of configuration settings. As users in different countries or regions may have various usage habits, some of the configuration items may correlate to countries or regions, e.g., time format or parameter units, the configuration items, which are the same according to the same country or region, are referred to herein as user maintenance items. The user maintenance items are types of configuration items.

Conventionally, patient monitors must be configured independently. For example, if multiple patient monitors need to use the same configuration settings, each respective patient monitor must be configured in turn. This process is complicated and prone to human error. Likewise, if the user maintenance items of different patient monitors need to have the same content, a user will need to separately configure the respective patient monitors, which results in repetitive operations, wasting time and energy.

In one embodiment, a method for transferring configuration settings may use a removable memory device, such as a USB flash drive, mobile hard drive, PCMCIA card, U disc, etc. Transferring configuration settings includes exporting and/or importing configuration settings using the removable memory device as the storage medium.

Exporting configuration settings includes storing the configuration settings exported from a patient monitor in a removable memory device. When configuration settings are being exported, the user can decide the specific configuration settings to be exported, which can be one or more sets of configuration settings, and/or one or more user maintenance items.

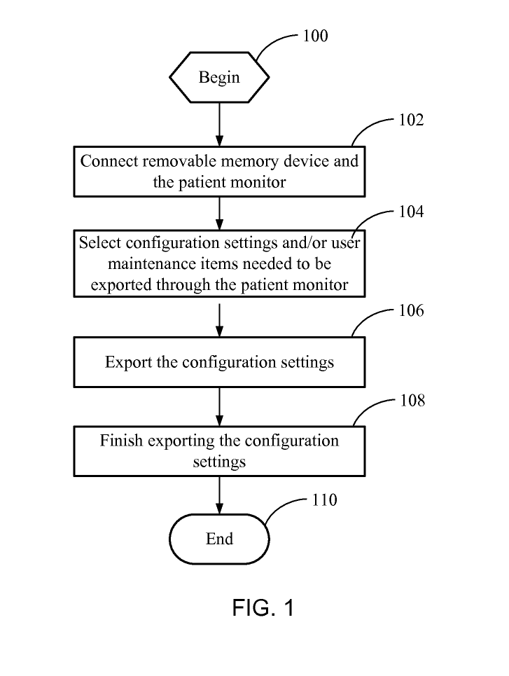

FIG. 1 is a flowchart of a process for exporting configuration settings according to one embodiment. The method includes:
100. beginning the process;
102. connecting the removable memory device and the patient monitor;
104. selecting configuration settings and/or user maintenance items needed to be exported through the patient monitor;
106. exporting the configuration settings;
108. finishing exporting the configuration settings; and
110. ending the process.

Importing configuration settings means to import configuration settings stored in the removable memory device to the patient monitor. During the importing process, the configuration settings stored in the removable memory device are listed on the patient monitor, and users can select the configuration settings that they want to import to the patient monitor. The user can select one or more sets of configuration settings and/or user maintenance items.

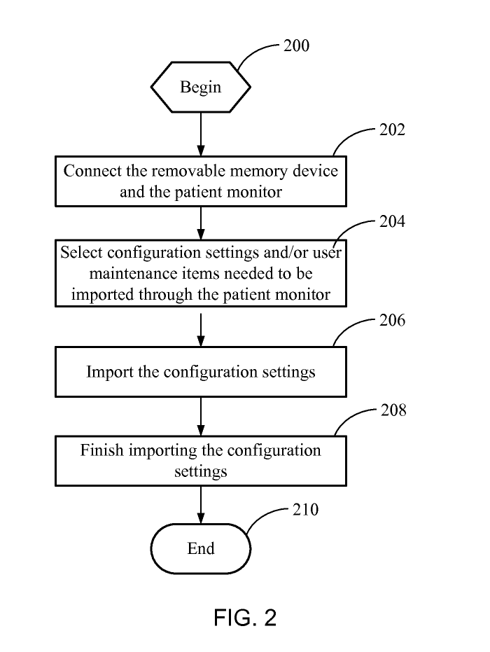

An exemplary flowchart of a method for importing configuration settings is shown in FIG. 2. The method includes:
200. beginning the process;
202. connecting the removable memory device and the patient monitor;
204. selecting configuration settings and/or user maintenance items needed to be imported to the patient monitor;
206. importing the configuration settings;
208. finishing importing the configuration settings; and
210. ending the process.

Operations of exporting and importing configuration settings can be implemented in a manner that don't interfere with the normal use of the patient monitors. Configuration settings can be transferred from one patient monitor to another through the removable memory device. Likewise, user maintenance items can be transferred from one patient monitor to another through the removable memory device. Using the removable memory device to transfer configuration settings or user maintenance items is very convenient, particularly when configuring multiple patient monitors with the same configuration settings and/or user maintenance items.

The process of selecting a set of configuration settings for a patient monitor is referred to herein as loading configuration settings. During this process, users may select a set of configuration settings from the removable memory device to be transferred. In addition, users may be able to browse the specific settings in a set of configuration settings before loading the configuration settings into the patient monitor.

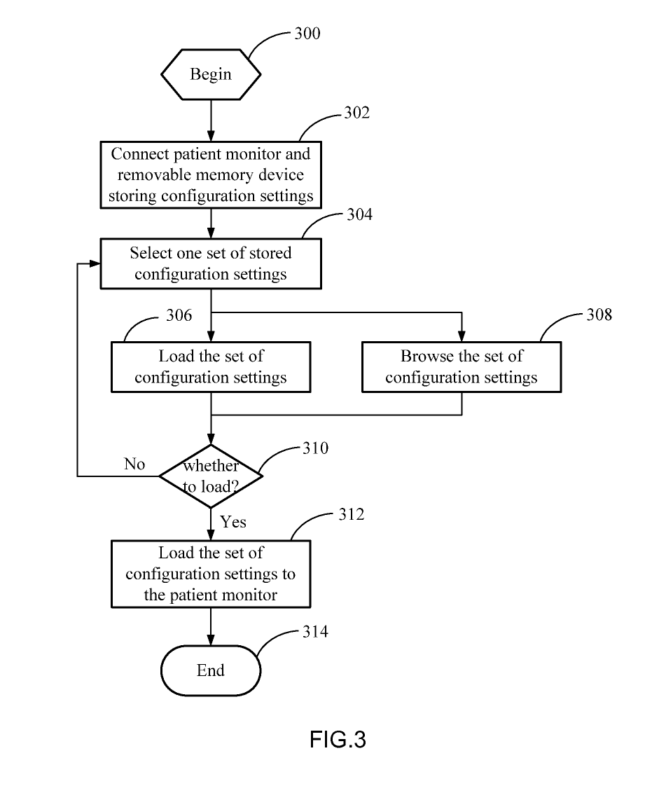

A flowchart of a method for loading configuration settings from the removable memory device is shown in FIG. 3. In one embodiment, the method includes:
300. beginning the process;
302. connecting the patient monitor and the removable memory device storing configuration settings;
304. selecting one set of configuration settings stored in the removable memory device;
306. loading the set of configuration settings;
308. browsing the set of configuration settings;
310. determining whether to load;
in response to an user command to not load, going to 304;
in response to a user command to load, going to 312;
312. loading the set of configuration settings to the patient monitor;
314. ending the process.

In one embodiment, the user can browse the specific context of selected configuration settings in the patient monitor after he or she has selected a set of configuration settings from the removable memory device. The set of configuration settings are loaded after being checked.

Typically, when loading configuration settings to the patient monitor, the user can only select the set of configuration settings he or she wants to load through the configuration setting name but can't see the specific context of configuration settings selected. If the user loads the set of configuration settings without knowing the specific context, the user may find the loaded set of configuration settings do not satisfy operating requirements, and it may increase risk to the patient because the set of configuration settings don't match the patient monitored. For example, among the set of configuration settings loaded, the alarm threshold of heart rate may be 150 bpm. However, for a particular patient, an alarm should be prompted to the health care staff if the patient's heart rate arrives at 120 bpm. Because of the incorrect setting, the health care staff will not be prompted at the proper time, and it will increase the patient's risk. In such a situation, if the user can browse the specific context of configuration settings and make sure the set of configuration settings selected can satisfy operating requirements before loading the configuration settings, these risks can be avoided.

In one embodiment, when the user browses the configuration settings, he or she can browse the configuration settings in the patient monitor and in the removable memory device without influencing the normal use of the patient monitor. After a set of configuration settings is selected, every specific context (e.g., parameter alarm threshold settings, parameter measurement settings, and patient monitor interface layout setting) of the selected set of configuration settings can be browsed through the patient monitor.

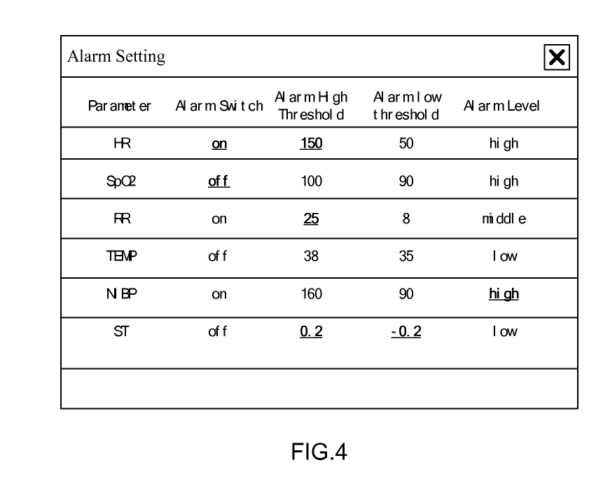

In addition, the difference between the selected set of configuration settings and the currently used configuration settings in the patient monitor may be marked in an distinctive way, e.g., underlining, highlighting, font, size, color, etc., so that it can be noticed easily by the user during browsing. FIG. 4 graphically represents an interface of alarm setting during browsing configuration settings. The underlined portions show the difference between the browsed set of configuration settings and the currently used set of configuration settings in the patient monitor. The context of configuration settings can be browsed directly in the patient monitor, allowing the user to know the specific context of the set of configuration settings, and lowering the risk caused by using an inappropriate set of configuration settings.

While browsing configuration settings, part or all of the configuration settings can be reviewed. There are other ways to browse the set of configuration settings currently used besides by menu. The browsed set of configuration settings and the currently used set of configuration settings are compared, then the difference may be marked by character font, graphic pattern, colors, and so on. In addition, it will be understood by those having skill in the art that many changes may be made to browse the set of configuration settings of the above-described embodiments without departing from the underlying principles of the invention.

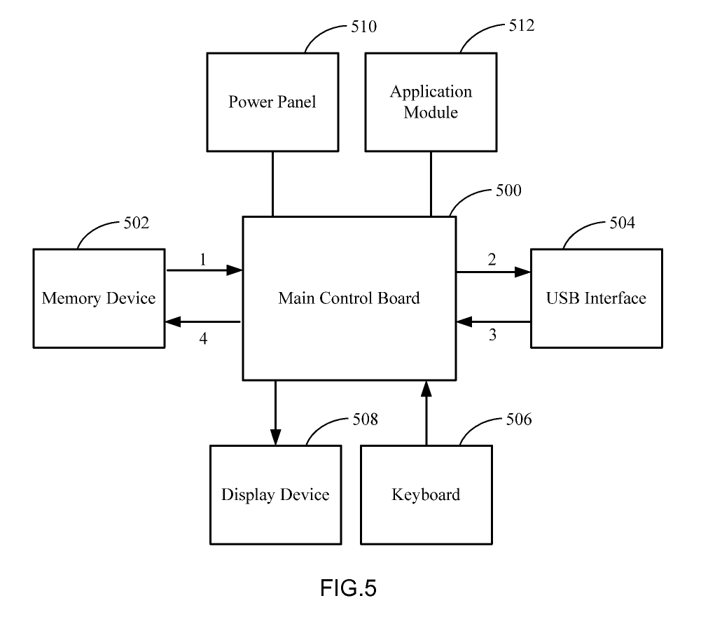

FIG. 5 is a block diagram of a patient monitor using the method described above according to one embodiment. The patient monitor includes a main control board 500 (which may include a processor), a memory device 502, a USB interface 504, a keyboard 506 (or other input device), a display device 508, a power panel 510, and an application module 512. The keyboard 506 is used to providing an application interface to users; the display device 508 is used to display the user operating interface; the configuration settings of the patient monitor is stored in the memory device 502; the USB interface 504 (or other type of interface) is used to connect a removable memory device; a set of configuration settings can be applied in the application module 512 after being loaded. The arrows with numbers in the FIG. 5 show operations to the configuration settings:

1. Loading configuration settings stored in the patient monitor's memory to be displayed on the patient monitor;

2. Exporting configuration settings stored in the patient monitor to the removable memory device;

3. Importing or loading the configuration settings stored in the removable memory device to the patient monitor;

4. Storing the configuration settings stored in the removable memory device to the memory of the patient monitor.

Exporting configuration settings by the removable memory device is the process of copying the configuration settings stored in the memory device of the patient monitor to the removable memory device. Importing configuration settings by the removable memory device is the process of copying the configuration settings stored in the removable memory device to the memory device of the patient monitor. Loading configuration settings is the process of reading a set of configuration settings from the memory device of the patient monitor and rewriting the currently used set of configuration settings. Loading configuration settings from a removable memory device is the process of reading a set of configuration settings from the removable memory device and rewriting the currently used set of configuration settings. Browsing configuration settings is the process of displaying a set of configuration settings by the display device, which can be the set of configuration settings stored in the patient monitor or in the removable memory device.

It will be understood by those having skill in the art that the above-described inventions can be used in other medical devices (e.g., diagnostic ultrasound devices) besides patient monitors. The system structures and compositions could be different in different devices, such as some devices do not include an application module; some devices could include other application interface (e.g., touch screen) instead of a keyboard for receiving commands.

The removable memory devices may include, for example, a removable memory card (e.g., Compact Flash (CF) card), USB flash drive, mobile hard disk, PCMCIA (PC) card, U disc, etc. Accordingly, interfaces should match the memory devices, such as CF card interfaces, PC card interfaces. It will be understood by those having skill in the art that many changes may be made to the transferring and loading configuration settings by other memory devices or interfaces memory devices without departing from the underlying principles of the invention.

A removable memory device according to one embodiment may include a memory device to store configuration settings of a medical device.

The removable memory device is used to realize at least one of the following operations after being connected to the medical device:

Exporting configuration settings stored in the removable memory device to the memory device of the medical device;

Exporting configuration settings stored in the memory device of the medical device to the removable memory device; and

Displaying configuration settings of the medical device stored in the removable memory device to the display device of the medical device.

In one embodiment, the difference between the configuration settings stored in the removable memory device and the currently used configuration settings in the medical device are shown by a display device during browsing the configuration settings stored in the removable memory device.

It will be understood by those having skill in the art that many changes may be made to the details of the above-described embodiments without departing from the underlying principles of the invention. The scope of the present invention should, therefore, be determined only by the following claims.

1. A medical device, comprising:
a memory, a display device, a removable memory device interface and a main control board; wherein the main control board is connected to the memory, the display device, and the removable memory device interface, and is configured to perform the following operations:
allowing a user to select a set of configuration settings and/or user maintenance items stored in a removable memory device coupled to the removable memory device interface;
displaying the selected set of configuration settings and/or user maintenance items on the display device; and
importing configuration settings and/or user maintenance items stored in the removable memory device to the memory of the medical device.

2. The medical device of claim 1, wherein displaying the selected set of configuration settings and/or user maintenance items comprises marking one or more of the configuration settings from the selected set of configuration settings that are different from one or more currently used configuration settings in the medical device.

3. The medical device of claim 2, wherein marking comprises using one or more of character font, highlighting, underlining, graphic pattern, or color to distinguish a configuration setting from the selected set of configuration settings that is different from a currently used configuration setting in the medical device.

4. The medical device of claim 1, wherein, after displaying the selected set of configuration settings, the main control board is configured to prompt the user to accept the selected set of configuration settings before importing them to the memory of the medical device.

5. The medical device of claim 1, wherein the main control board is configured to selectively export one or more configuration settings and/or user maintenance items in the memory of the medical device to the removable memory device.

6. The medical device of claim 1, wherein the medical device is a patient monitor.

7. The medical device of claim 1, wherein the medical device is a diagnostic ultrasound device.

8. The medical device of claim 1, wherein the removable memory device is selected from the group consisting of a removable memory card, flash drive, mobile hard disk, PCMCIA card, and a U disc.

9. The medical device of claim 1, wherein at least one user maintenance item relates to a setting for a particular country or region.

10. The medical device of claim 1, wherein at least one user maintenance items for a particular country or region is a time format or parameter unit.

11. A method for transferring configuration settings and/or user maintenance items, comprising:
allowing a user to select a set of configuration settings and/or user maintenance items stored in a removable memory device coupled to a medical device;
displaying the selected set of configuration settings and/or user maintenance items on a display device; and
importing the selected set of configuration settings and/or user maintenance items stored in the removable memory device to the medical device.

12. The method of claim 11, wherein displaying the selected set of configuration settings comprises marking one or more of the configuration settings from the selected set of configuration settings that are different from one or more currently used configuration settings in the medical device.

13. The method of claim 12, wherein marking comprises using one or more of character font, highlighting, underlining, graphic pattern, or color to distinguish a configuration setting from the selected set of configuration settings that is different from a currently used configuration setting in the medical device.

14. The method of claim 11, wherein the method further comprises, after displaying the selected set of configuration settings, prompting the user to accept the selected set of configuration settings before importing them to the memory of the medical device.

15. The method claim 11, wherein the method further comprises selectively exporting one or more configuration settings and/or user maintenance items in the memory of the medical device to the removable memory device.

16. The method of claim 11, wherein the medical device comprises a patient monitor.

17. The method of claim 11, wherein the medical device comprises a diagnostic ultrasound device.

18. The method claim 11, wherein the removable memory device is selected from the group consisting of a removable memory card, flash drive, mobile hard disk, PCMCIA card, and a U disc.

19. The method of claim 11, wherein at least one user maintenance item relates to a setting for a particular country or region.

20. The method of claim 11, wherein at least one user maintenance item for a particular country or region is a time format or parameter unit.
